(19) United States
(12) Patent Application Publication
(10) Pub. No.: US 2009/0227867 A1
(43) Pub. Date: Sep. 10, 2009

(54) ULTRASONOGRAPH

(75) Inventors: Takao Suzuki, Kanagawa (JP); Hisashi Hagiwara, Kanagawa (JP); Makoto Kato, Kanagawa (JP); Yoshinao Tan-naka, Kanagawa (JP); Hiroshi Kanai, Miyagi (JP); Hideyuki Hasegawa, Miyagi (JP)

Correspondence Address: MARK D. SARALINO (PAN);RENNER, OTTO, BOISSELLE & SKLAR, LLP, 1621 EUCLID AVENUE, 19TH FLOOR, CLEVELAND, OH, 44115, US

(73) Assignees: MATSUSHITA ELECTRIC INDUSTRIAL CO., LTD., Kadoma-shi, Osaka (JP); TOHOKU UNIVERSITY, Sendai-shi, Miyagi (JP)

(21) Appl. No.: 12/097,142

(22) PCT Filed: Dec. 13, 2006
(86) PCT No.: PCT/JP2006/324855

§ 371 Date: Sep. 10, 2008

(30) Foreign Application Priority Data
Dec. 15, 2005 (JP) 2005-362057

(51) Int. Cl. A61B 8/14

(52) U.S. Cl. 600/443

(57) ABSTRACT

An ultrasonic diagnostic apparatus according to the present invention is designed to measure a vascular wall. The apparatus includes: a transmitting section that drives a probe to transmit an ultrasonic wave toward a subject including a blood vessel; a receiving section that receives an ultrasonic echo, produced by getting the ultrasonic wave reflected by the subject, at the probe to generate a received signal; a tomographic image processing section for generating an image signal based on the received signal to present the tomographic image of the subject on a display section; a first borderline generating section for determining a first type of borderline based on the tomographic image presented on the display section; and a second borderline generating section for generating at least one borderline of a second type by translating the first type of borderline.

### TECHNICAL FIELD

The present invention relates to an ultrasonic diagnostic apparatus for use in medical applications and more particularly relates to an ultrasonic diagnostic apparatus for measuring a vascular wall.

### BACKGROUND ART

An ultrasonic diagnostic apparatus is used to monitor an internal tissue of a subject by irradiating him or her with an ultrasonic wave and analyzing the information contained in its echo signal. For example, a conventional ultrasonic diagnostic apparatus that has been used extensively converts the intensity of an echo signal into its associated pixel luminance, thereby presenting the subject's structure as a tomographic image. In this manner, the internal structure of the subject can be known. The ultrasonic diagnostic apparatus can be used to make a noninvasive checkup on an internal tissue of a subject, and therefore, is now an indispensable device at any clinical spot along with X-ray CT and MRI.

Recently, the number of people suffering from arteriosclerosis has been on the rise and carotid echo has been carried out more and more often using an ultrasonic diagnostic apparatus to diagnose arteriosclerosis. It is known that the carotid artery has a three-layer structure consisting of intima, media and adventitia that are stacked in this order. In carrying out a carotid echo, the combined thickness of the intima and the media (or intima-media thickness, which will be abbreviated herein as “IMT”) is measured and used as an index to arteriosclerosis. According to Non-Patent Document No. 1, if the IMT is 1.1 mm or more, the carotid is determined to have abnormally thickened. The IMT is manually measured with a length measuring function on a tomographic image, which is usually provided for any ultrasonic diagnostic apparatus. The IMT may be measured sometimes at only one spot and sometimes at multiple spots. In the latter case, the maximum or average of those multiple measured values may be used as an index. FIG. 12 shows how the IMT of a carotid artery is measured with a conventional ultrasonic diagnostic apparatus. Specifically, FIG. 12 shows a tomographic image as viewed on a plane that is parallel to the axis of the vascular wall (i.e., a vertical cross section of the vascular wall). In FIG. 12, the six + signs indicate manually set points. At the central pair of set points indicated by “1”, the distance between the two + signs is 0.5 mm. On the other hand, at each of the other two pairs of set points indicated by “2” and “3”, the distance between the two + signs is 0.4 mm.

Meanwhile, as an alternative method for diagnosing arteriosclerosis, some people are attempting recently to track the motion of a subject's tissue more precisely and evaluate the strain and the elasticity, viscosity or any other attribute property of the tissue mainly by analyzing the phase of the reflected echo signal.

Patent Document No. 1 discloses a method for tracking precisely the motions of two very small regions on a vascular wall in one cardiac cycle, sensing a slight variation in the thickness (i.e., the magnitude of strain) that is superposed on a significant oscillation due to the heartbeat and calculating a local elasticity based on the magnitude of strain and blood pressure difference. Patent Document No. 1 also discloses an apparatus for displaying a spatial distribution of elasticities as an image. To track those very small regions on a vascular wall, a phase difference tracking method as disclosed in Patent Document No. 2 is used. Hereinafter, a method for tracking a subject's tissue as disclosed in Patent Document No. 1 will be described with reference to FIGS. 13(a) and 13(b). As shown in FIG. 13(a), an ultrasonic wave is transmitted from a probe 101 toward the blood vessel 111 of a subject 110 and an echo reflected from the blood vessel 111 is received at the probe 101. Two measuring points A and B are set on the vascular wall and the signals received from those measuring points A and B are analyzed by the method disclosed in Patent Document No. 2, thereby tracking the motions of the measuring points A and B.

The blood vessel 111 repeatedly contracts and dilates through cardiac cycles. More specifically, the blood vessel 111 dilates rapidly in a systolic phase but contracts slowly in a diastolic phase. FIG. 13(b) shows the tracking waveforms TA and TB of the measuring points A and B along with an electrocardiographic complex ECG. As the blood vessel 111 dilates, the measuring points A and B move rapidly. But after that, the measuring points A and B slowly go back to their original locations. The difference between the tracking waveforms TA and TB is represented as a waveform W showing a variation in thickness between the measuring points A and B. Supposing the variation in the thickness variation waveform is ΔW and the reference thickness between the measuring points during initialization is Ws, the magnitude of strain ε between the measuring points A and B is calculated by the following Equation (1):

ε=ΔW/Ws (1)

Supposing the blood pressure difference at this time is ΔP, the radial elasticity Er between the measuring points A and B is given by the following Equation (2):

Er=ΔP/ε=ΔP·Ws/ΔW (2)

Therefore, by measuring the elasticity Er for multiple spots on a tomographic image, an image representing the distribution of elasticities can be obtained.

Non-Patent Document No. 2 discloses a method for calculating the circumferential elasticity of a blood vessel, representing a more accurate physical property than the radial elasticity thereof. According to Non-Patent Document No. 2, the circumferential elasticity is given by the following Equation (3):

$$\begin{matrix}E θ = - ( 1 / 2 ) \cdot ( r 0 / h 0 + 1 ) \cdot ( Δ P / ɛ ) \\ = - ( 1 / 2 ) \cdot ( r 0 / h 0 + 1 ) \cdot Er\end{matrix} \qquad ( 3 )$$

where h0 is the initial radial thickness of the overall vascular wall and r0 is initial radius of the blood vessel.

FIG. 14 illustrates a picture to be presented on a monitor screen when the artery is inspected with an ultrasonic diagnostic apparatus. On the screen, an elasticity image 201 representing the distribution of elasticities is superimposed on the posterior wall portion 230 (i.e., a portion of the vascular wall that is more distant from the skin surface) of a tomographic image 200.

In the prior art, to calculate the initial thickness h0 and initial radius r0 of the overall vascular wall, the borderline 203 between the vascular flow 207 and the intima 221 of the anterior wall (i.e., a portion of the vascular wall closer to the skin surface) 220, the borderline 204 between the vascular flow 207 and the intima 231 of the posterior wall 230, and the borderline 206 between the adventitia 233 of the posterior wall 230 and its surrounding tissue are drawn manually. More specifically, by sensing the boundary between the area of the vascular flow 207 and the area of the intima 221 of the anterior wall 220 that are presented on the monitor screen based on the shades of the tomographic image reflection intensity scale 202, the operator traces the borderline with the cursor 210 on the monitor screen, thereby determining the borderline 203. By repeatedly performing similar operations, the other borderlines 204 and 206 can also be drawn.
Patent Document No. 1: Japanese Patent Application Laid-Open Publication No. 2000-229078
Patent Document No. 2: Japanese Patent Application Laid-Open Publication No. 10-5226
Non-Patent Document No. 1: Hiroshi Furuhata, Carotid Echo, Vector Core Inc., 2004, ISBN 4-938372-88-6
Non-Patent Document No. 2: Hideyuki Hasegawa, Hiroshi Kanai et al., “Evaluation of regional elastic modulus of cylindrical shell with non-uniform wall thickness”, J. Med. Ultrasonics, Vol. 28, No. 1 (2001), pp. J3 to J13

### DISCLOSURE OF INVENTION

### Problems to be Solved by the Invention

However, as there are multiple borderlines to draw, it will take a while to establish all of those borderlines. Also, since the initial thickness h0 and initial radius r0 of the overall vascular wall are calculated based on the borderlines that have been drawn in this manner, the borderlines should be determined accurately. For that reason, it would impose a heavy burden on the operator to determine the borderlines by moving the cursor by him- or herself.

In order to overcome the problems described above, the present invention has an object of providing an ultrasonic diagnostic apparatus that can alleviate the burden on the operator.

### Means for Solving the Problems

An ultrasonic diagnostic apparatus according to the present invention is designed to measure a vascular wall. The apparatus includes: a transmitting section that drives a probe to transmit an ultrasonic wave toward a subject including a blood vessel; a receiving section that receives an ultrasonic echo, produced by getting the ultrasonic wave reflected by the subject, at the probe to generate a received signal; a tomographic image processing section for generating an image signal based on the received signal to present the tomographic image of the subject on a display section; a first borderline generating section for determining a first type of borderline based on the tomographic image presented on the display section; and a second borderline generating section for generating at least one borderline of a second type by translating the first type of borderline.

In one preferred embodiment, the ultrasonic diagnostic apparatus further includes a user interface that allows an operator to specify his or her desired location on the tomographic image.

In this particular preferred embodiment, the second borderline generating section generates the second type of borderline by translating the first type of borderline to the location that has been specified by the operator with the user interface in accordance with information about the first type of borderline.

In a specific preferred embodiment, the first borderline generating section stores, as the information about the first type of borderline, information about a line segment that has been drawn on the tomographic image by the operator with the user interface.

In a more specific preferred embodiment, the first borderline generating section generates either a line segment or a polygon as the first type of borderline at the location that has been specified by the operator.

In another preferred embodiment, the first type of borderline is located on at least one boundary that is selected from a group of boundaries consisting of boundaries between the intima of the anterior and posterior walls of the blood vessel and vascular flow, boundaries between the media and adventitia of the anterior and posterior walls of the blood vessel, and boundaries between the adventitia of the anterior and posterior walls of the blood vessel and their surrounding tissue. The second type of borderline is located on at least another one of the group of boundaries.

In this particular preferred embodiment, the first borderline generating section detects the boundary between the intima of the anterior or posterior wall of the blood vessel and the vascular flow based on the image signal and determines the first type of borderline on the boundary detected.

In a specific preferred embodiment, the first type of borderline is located on the boundary between the intima of the anterior or posterior wall and the vascular flow and the second type of borderline is located on the boundary between the media and adventitia thereof. The intima-media thickness of the vascular wall is calculated by reference to the first and second types of borderlines.

In a more specific preferred embodiment, the ultrasonic diagnostic apparatus further includes: a tissue tracking section for tracking the motion of a tissue of the subject based on the received signal; and an attribute value calculating section that receives information about the blood pressure of the subject and calculates an attribute value of the tissue based on the motion of the tissue tracked. The attribute value of at least one tissue, which is selected from the group consisting of the intima, media and adventitia of the blood vessel, is calculated with respect to the first and second types of borderlines.

In this particular preferred embodiment, the first type of borderline is located on the boundary between the intima and the vascular flow and the second type of borderline is located between the adventitia and the surrounding tissue. By calculating the radius of the blood vessel and the thickness of the vascular wall by reference to the first and second types of borderlines, a circumferential elasticity is calculated as the attribute value.

A method for determining the boundaries of a vascular wall using an ultrasonic diagnostic apparatus according to the present invention includes the step of generating a second type of borderline by translating a first type of borderline on a tomographic image of a subject's blood vessel that has been produced by transmitting and receiving an ultrasonic wave. The first type of borderline is determined so as to be located on at least one boundary that is selected from a group of boundaries consisting of boundaries between the intima of the anterior and posterior walls of the blood vessel and vascular flow, boundaries between the media and adventitia of the anterior and posterior walls of the blood vessel, and boundaries between the adventitia of the anterior and posterior walls of the blood vessel and their surrounding tissue. The second type of borderline is located on at least another one of the group of boundaries.

In one preferred embodiment, the method further includes the step of storing, as information about the first type of borderline, information about a line segment that has been drawn on the tomographic image by the operator using a user interface. The step of generating the second type of borderline includes generating the second type of borderline based on the stored information about the first type of borderline.

In another preferred embodiment, the method further includes the step of generating either a line segment or a polygon as the first type of borderline at the location that has been specified on the tomographic image of the blood vessel by the operator using the user interface.

In still another preferred embodiment, the method further includes the step of detecting the boundary between the intima of the anterior or posterior wall of the blood vessel and the vascular flow based on an image signal representing the tomographic image and determining the first type of borderline on the boundary detected.

### Effects of the Invention

According to the present invention, time and trouble to determine the respective boundaries of a vascular wall can be saved and measurements by an ultrasonic diagnostic apparatus can be done in a shorter time.

### BRIEF DESCRIPTION OF DRAWINGS

FIG. 1(a) is a block diagram illustrating a configuration for an ultrasonic diagnostic apparatus according to the present invention and FIG. 1(b) is a block diagram illustrating the configuration of its core section.

FIG. 2 is a flowchart showing a procedure for determining borderlines using the ultrasonic diagnostic apparatus shown in FIG. 1.

FIG. 3 illustrates an exemplary picture to be presented on the monitor screen when the procedure shown in FIG. 2 is adopted.

FIGS. 4(a) through 4(c) illustrate an exemplary procedure for generating a borderline by translating another borderline.

FIGS. 5(a) through 5(c) illustrate another exemplary procedure for generating a borderline by translating another borderline.

FIGS. 6(a) through 6(c) illustrate still another exemplary procedure for generating a borderline by translating another borderline.

FIG. 7 is a flowchart showing another procedure for determining borderlines using the ultrasonic diagnostic apparatus shown in FIG. 1.

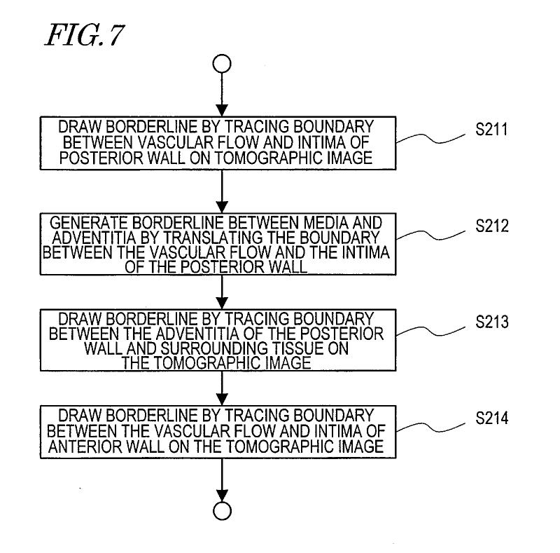

FIG. 8 illustrates an exemplary picture to be presented on the monitor screen when the procedure shown in FIG. 7 is adopted.

FIG. 9 is a flowchart showing still another procedure for determining borderlines using the ultrasonic diagnostic apparatus shown in FIG. 1.

FIG. 10 illustrates an exemplary picture to be presented on the monitor screen when the procedure shown in FIG. 8 is adopted.

FIG. 11 is a flowchart showing yet another procedure for determining borderlines using the ultrasonic diagnostic apparatus shown in FIG. 1.

FIG. 12 shows an exemplary plane on which an IMT is measured with a conventional ultrasonic diagnostic apparatus.

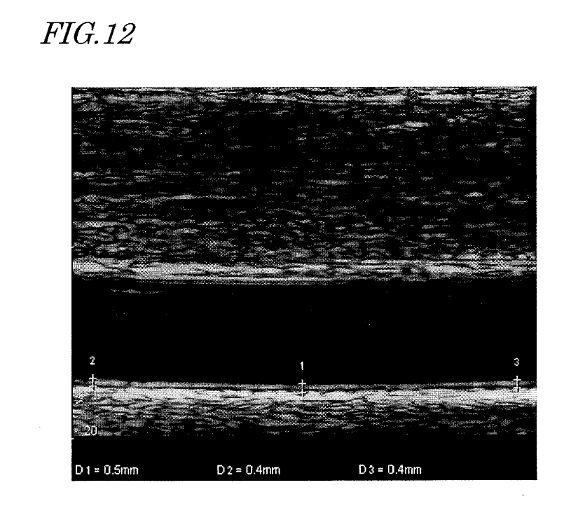

FIG. 13(a) illustrates a procedure for calculating the magnitude of strain based on the tracking waveforms at multiple measuring points using a conventional ultrasonic diagnostic apparatus and FIG. 13(b) shows the tracking waveforms at those measuring points.

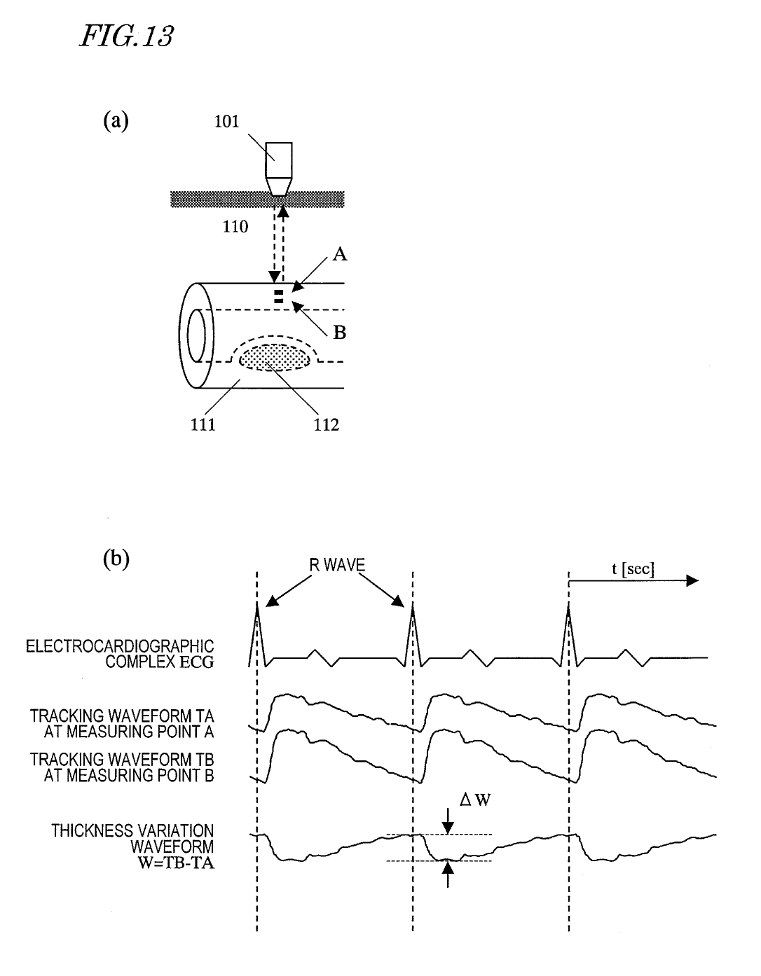

FIG. 14 illustrates a typical elasticity image presented on the screen of a conventional ultrasonic diagnostic apparatus.

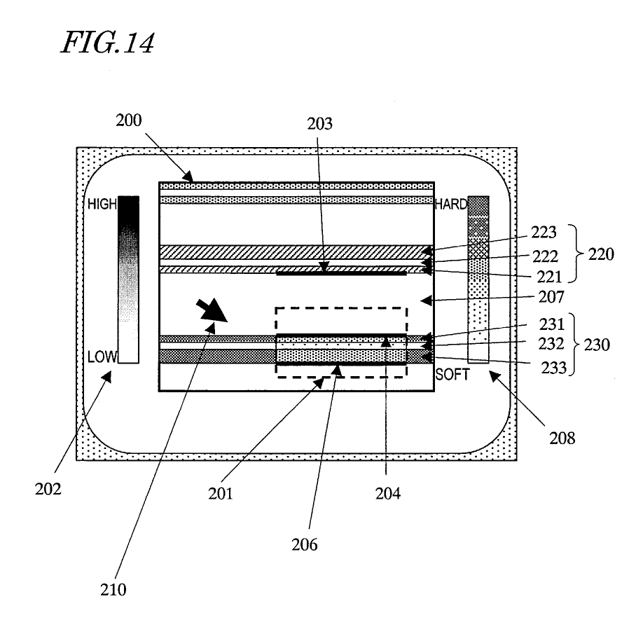

### DESCRIPTION OF REFERENCE NUMERALS

100 control section
101 probe
102 transmitting section
103 receiving section
104 tomographic image processing section
105 tissue tracking section
1≢image synthesizing section
107 monitor
108 elasticity calculating section
111 blood pressure manometer
112 blood pressure manometer controlling and blood pressure value input section
120, 121 memory
130 user interface
150 borderline determining section
151 borderline storage section
152 second borderline generating section
153 first borderline generating section
200 tomographic image
202 tomographic image reflection intensity scale
203 vascular flow-intima borderline on anterior wall of blood vessel
204 vascular flow-intima borderline on posterior wall of blood vessel
205 media-adventitia borderline on posterior wall of blood vessel
206 adventitia-surrounding tissue borderline on posterior wall of blood vessel

### BEST MODE FOR CARRYING OUT THE INVENTION

Hereinafter, preferred embodiments of an ultrasonic diagnostic apparatus according to the present invention will be described with reference to the accompanying drawings. An ultrasonic diagnostic apparatus according to the present invention is used to determine the shape or an attribute value of a vascular wall. In the preferred embodiments to be described below, a boundary between the vascular flow and the intima of the anterior wall of a blood vessel and boundaries between the vascular flow and the intima, between the media and adventitia, and between the adventitia and a surrounding tissue of the posterior wall of a blood vessel are supposed to be determined. However, the present invention is in no way limited to those specific preferred embodiments but is also applicable for use to determine a boundary between the media and adventitia of the anterior wall and a boundary between the adventitia and a surrounding tissue of the anterior wall.

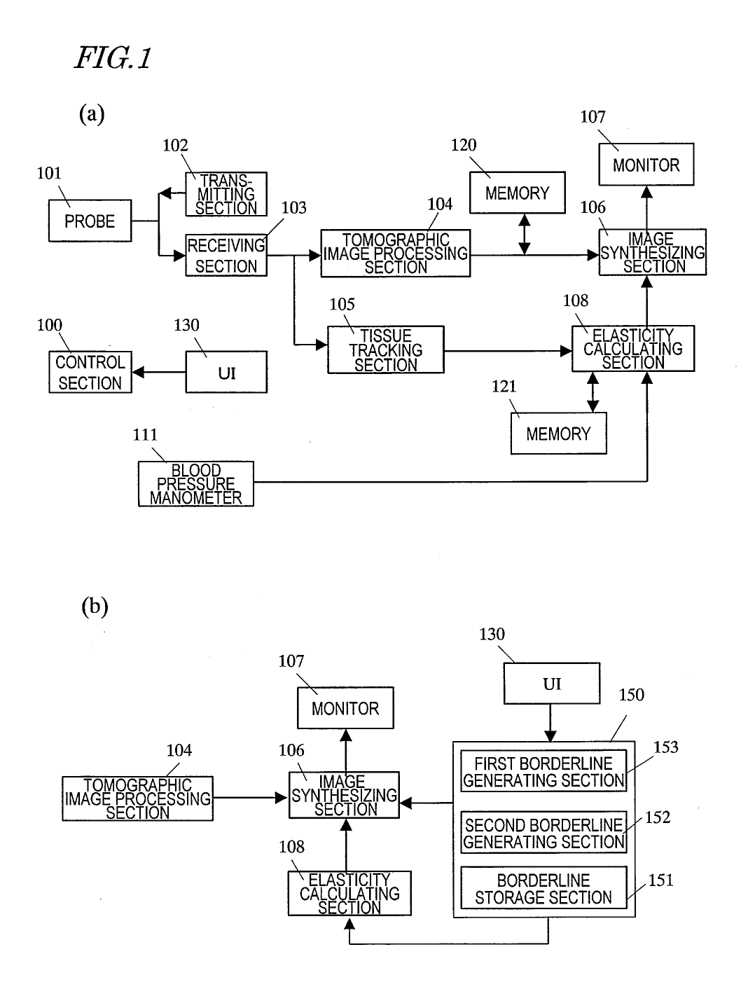

FIG. 1(a) is a block diagram illustrating a configuration for an ultrasonic diagnostic apparatus as a preferred embodiment of the present invention. The ultrasonic diagnostic apparatus includes a transmitting section 102, a receiving section 103, a tomographic image processing section 104, a tissue tracking section 105, an image synthesizing section 106, and an elasticity calculating section 108. The apparatus further includes a control section 100 for controlling all of these components and a user interface 130.

The user interface 130 is an input device that accepts an operator's command such as a keyboard, a trackball, a switch or a button. The operator's command that has been entered through the user interface 130 is input to the control section 100. The control section 100 may be implemented as a microcomputer, for example, and controls all of those components in accordance with the operator's commands. In addition, as will be described in detail later with reference to FIG. 1(b), the control section 100 includes a borderline determining section 150 for determining borderlines indicating boundaries between the tissues of a vascular wall. It should be noted that although the control section 100 actually exchanges signals with the respective blocks shown in FIG. 1(a), lines indicating exchange of those signals are not shown in FIG. 1(a) to avoid complications.

In accordance with the instruction given by the control section 100, the transmitting section 102 generates a high-voltage transmission signal that drives the probe 101 at a specified timing. The probe 101 converts the transmission signal that has been generated by the transmitting section 102 into an ultrasonic wave and sends out the ultrasonic wave toward a subject, and also detects an ultrasonic echo that has been reflected by an internal organ of the subject and converts the echo into an electrical signal. A number of piezoelectric transducers are arranged in the probe 101. By changing the piezoelectric transducers to use and the timing to apply a voltage to the piezoelectric transducers, the probe 101 controls the angle of deviation and focus of the ultrasonic waves to transmit and receive. The receiving section 103 amplifies the electrical signal generated by the probe 101 and outputs a received signal. Also, the receiving section 103 detects only an ultrasonic wave that has come from a particular position (associated with the focus) or direction (associated with the angle of deviation).

The tomographic image processing section 104 includes a filter, a detector, and a logarithmic amplifier, and analyzes mainly the amplitude of the received signal, thereby generating an image signal representing a tomographic image of the subject. The tissue tracking section 105 includes a magnitude of displacement calculating section for analyzing the phase difference between the received signals and calculating the magnitude of displacement of the subject's tissue in the ultrasonic wave transmitting and receiving directions, and a tracking location calculating section that calculates the new location by adding the magnitude of displacement to the original location. The tissue tracking section 105 tracks the motion of the subject's tissue in the ultrasonic wave transmitting and receiving directions.

The elasticity calculating section 108 calculates an attribute property value such as the magnitude of strain or the elasticity. Specifically, first, the elasticity calculating section 108 calculates the magnitude of strain based on the motion of the subject's tissue tracked. The elasticity calculating section 108 also receives information about the blood pressure of the subject from the blood pressure manometer 111. Furthermore, as will be described in detail later, the elasticity calculating section 108 receives information about first and second types of borderlines that have been determined by the borderline determining section 150, calculates the distance between the borderlines in the subject, and also calculates the radius of the blood vessel and the thickness of the vascular wall. And based on the magnitude of strain, the information about the blood pressure, the radius of the blood vessel, and the thickness of the vascular wall, the elasticity calculating section 108 calculates the radial and circumferential elasticities and outputs them as numerical values or a two-dimensional distribution image.

The image synthesizing section 106 synthesizes together the tomographic image and at least one of the elasticity image and the elasticity values and outputs the synthesized image to the monitor 107. The memory 121 stores the location tracking information (i.e., the motion of the subject's tissue) and/or the magnitude of strain. With the transmission and reception of the ultrasonic waves by the probe 101 suspended (which will be referred to herein as a “freeze state”), the information stored in the memory 121 is read to re-calculate the elasticity values. On the other hand, the memory 120 stores the image signal. In the freeze state, the image signal representing the tomographic image is read synchronously with the elasticity.

Next, the borderline determining section 150 will be described in detail. FIG. 1(b) is a block diagram illustrating the core section of the present invention. The borderline determining section 150 draws a first type of borderline based on the tomographic image presented on the monitor 107 and then generates at least one borderline of a second type by translating the first type of borderline thus determined.

For that purpose, the borderline determining section 150 includes a first borderline generating section 153, a second borderline generating section 152 and a borderline storage section 151. The first borderline generating section 153 draws a first type of borderline based on the tomographic image and stores information about the first type of borderline in the borderline storage section 151. This first type of borderline is a line segment to be drawn as a cursor trace by allowing the operator to move the cursor using the user interface 130 such as a mouse while the tomographic image of the subject is being presented on the monitor 107.

The first borderline generating section 153 may regard either a line segment or a polygon, which connects two or more points that have been specified by the operator with the mouse clicked, as the first type of borderline and store its information. Alternatively, the first borderline generating section 153 may obtain a tropic by connecting a plurality of points specified by the operator and regard the tropic as the first type of borderline. Still alternatively, the first borderline generating section 153 may also obtain a spline function that passes a plurality of points specified by the operator and use the spline function as the first type of borderline. The number of borderlines of the first type does not have to be one. If necessary, multiple borderlines may be drawn as borderlines of the first type.

In accordance with the information about the first type of borderline that is stored in the borderline storage section 151, the second borderline generating section 152 translates the first type of borderline to the location specified by the operator, thereby generating a second type of borderline. Optionally, if multiple borderlines of a first group have been determined and stored by the first borderline generating section 153, then the user interface 130 may select one of those borderlines of the first group stored.

The information about the first and second types of borderlines is output to the elasticity calculating section 108. Based on these pieces of information, the elasticity calculating section 108 figures out the distance between the borderlines in the subject and uses the distance thus obtained to calculate the elasticity.

Such functions of the borderline determining section 150 will be described more fully with reference to a tomographic image presented on the monitor 107.

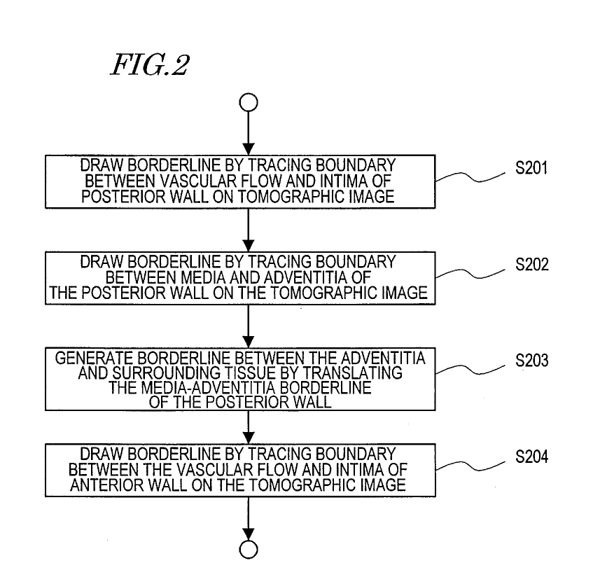

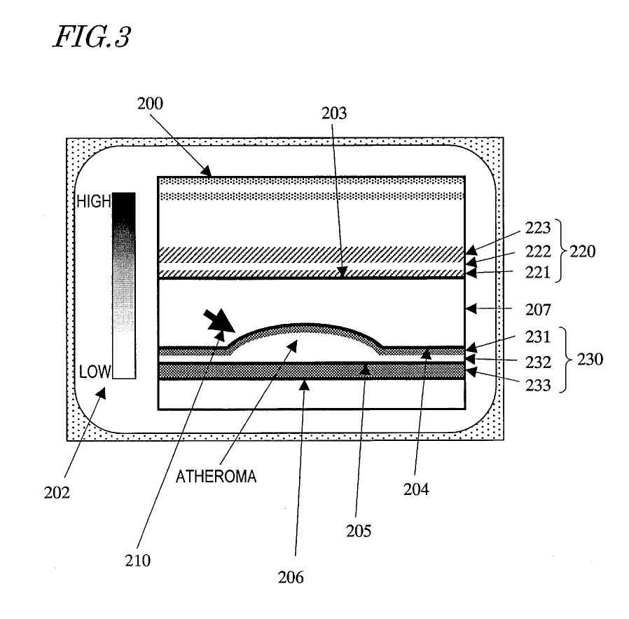

FIG. 2 is a flowchart showing the procedure of determining borderlines between the respective tissues of a vascular wall using a tomographic image that is viewed on a plane parallel to the axis of the blood vessel. First, the ultrasonic diagnostic apparatus transmits and receives ultrasonic waves to/from an internal organ of a subject, thereby getting a tomographic image of his or her vascular wall. As shown in FIG. 3, a tomographic image 200 that has been obtained properly shows clearly intima 221, media 222 and adventitia 223 in the anterior wall 220 and another intima 231, another media 232 and another adventitia 233 in the posterior wall 230. FIG. 3 shows a case where a boss called “atheroma” has been produced between the intima 231 and media 232 of the posterior wall 230 due to the congestion of fat or cholesterol there. To determine the borderlines between the respective tissues of the vascular wall in the state shown in this tomographic image 200, the ultrasonic diagnostic apparatus is made to enter the freeze state.

First, in Step S201, by moving a cursor 210, which is displayed on the screen of the monitor 107, using the user interface 130 such as a trackball or a mouse provided for the ultrasonic diagnostic apparatus, the boundary between the vascular flow 207 and the intima 231 of the posterior wall 230 on the tomographic image 200 is traced with the cursor 210. As a result, a line segment is drawn as a borderline 204 between the vascular flow 207 and the intima 231 of the posterior wall 230 on the screen. The borderline storage section 151 stores information about the borderline 204 as a first type of borderline.

Next, in Step S202, a borderline 205 is determined in a similar manner between the media 232 and the adventitia 233 of the posterior wall 230. The borderline storage section 151 stores information about the borderline 205 as another borderline of the first type.

Subsequently, in Step S203, the second borderline generating section 152 translates the borderline 205 to the location specified by the operator in accordance with the information about the borderline 205 that is stored in the borderline storage section 151, thereby generating a borderline 206 on the boundary between the adventitia 233 of the posterior wall 230 and its surrounding tissue.

Finally, in Step S204, by moving the cursor 210 on the screen of the monitor 107, the boundary between the vascular flow 207 and the intima 221 of the anterior wall 220 on the tomographic image 200 is traced with the cursor 210, thereby drawing a borderline 203 between the vascular flow 207 and the intima 221 of the anterior wall 220. The borderline storage section 151 stores information about the borderline 203 as another borderline of the first type. By following such a procedure, the burden imposed on the operator by tracing boundaries and the time it takes to determine the borderlines can be reduced by one step.

In the processing step S203, the borderline can be generated by any of various image processing techniques such as copying a line segment on a computer screen and pasting it at a predetermined location.

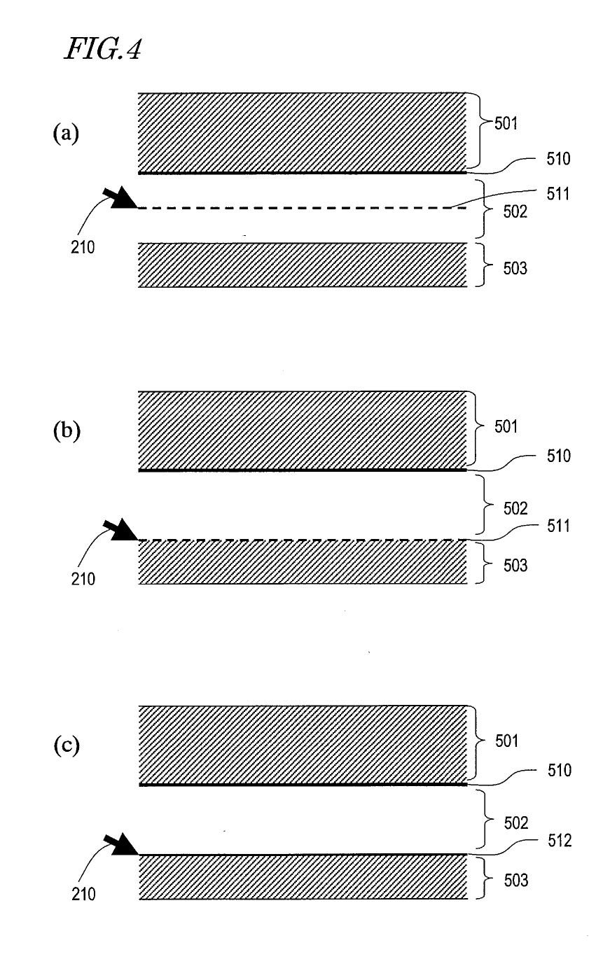

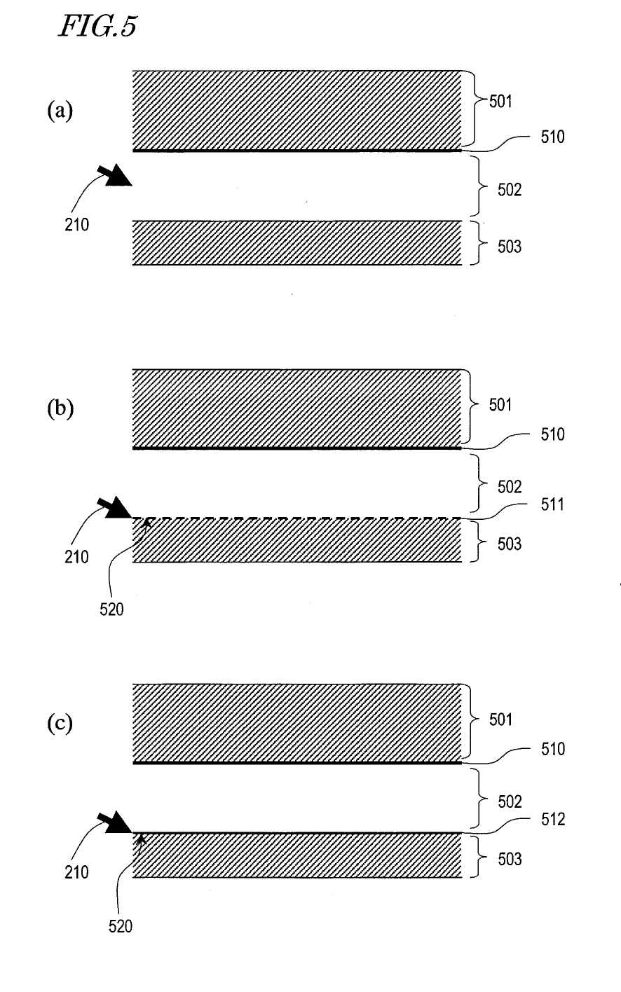

FIGS. 4(a) through 4(c) and FIGS. 5(a) through 5(c) show examples of such procedures. In these drawings, regions 501, 502 and 503 schematically represent mutually different tissues.

First, as shown in FIG. 4(a), a borderline 510 is drawn as a first type of borderline on the boundary between the tissues 501 and 502. This borderline 510 is obtained as a line segment that has been drawn as a trace of the cursor 210 by allowing the operator to move the cursor 210 along the boundary between the tissues 501 and 502 using the user interface 130. By determining the borderline 510 in this manner, information about the borderline 510 is stored in the borderline storage section 151. Alternatively, the operator may also determine the borderline 510 by specifying multiple locations with the cursor 210 and generating a line or a polygon passing all of those specified locations as described above. Still alternatively, the borderline 510 may also be determined by generating either a tropic by a minimum square method or a spline function with respect to the multiple locations specified.

After the borderline 510 has been determined in this manner, the second borderline generating section 152 generates a borderline 511, having the same shape and the same direction as the borderline 510, at the tip of the cursor 210 in accordance with the operator's command that has been entered through the user interface 130. For example, by entering a “copy” command through the user interface 130, the borderline 511 may be generated and presented on the monitor screen. This borderline 511 is generated based on the information about the borderline 510 stored in the borderline storage section 151 and the location information of the cursor 210. When the operator moves the cursor 210, the borderline 511 is also translated.

Next, as shown in FIG. 4(b), the operator moves the cursor 210 to a location where a borderline should be generated. In the example shown in FIG. 4(b), the cursor 210 is located on the boundary between the regions 502 and 503 to determine a borderline on the boundary between the regions 502 and 503.

When the cursor 210 is moved to a particular location in this manner, the borderline 510 is translated to that location, thereby generating and displaying a borderline 511 there.

Subsequently, if the operator inputs a confirmation command such as a paste command through the user interface 130, a borderline 512 is established at that location as shown in FIG. 4(c). In this manner, the borderline 512, generated by translating the borderline 510 that has been drawn by the operator, is determined as the second type of borderline.

Alternatively, another procedure may also be adopted. First, as shown in FIG. 5(a), a borderline 510 is drawn as a first type of borderline on the boundary between the tissues 501 and 502. The borderline 510 is drawn in the same way as already described with reference to FIG. 4(a).

After the borderline storage section 151 stores information about the borderline 510 as a first type of borderline, the apparatus changes into a “copy” mode in accordance with an operator's command that has been entered through the user interface 130. According to this procedure, the operator can move the cursor 210 to his or her desired location with the user interface 130 without displaying a copy of the borderline 510 on the screen.

As shown in FIG. 5(b), the operator has moved the cursor 210 to the location where a borderline should be generated. In the example shown in FIG. 5(b), the cursor 210 is located on the boundary between the regions 502 and 503 to determine a borderline on the boundary between the regions 502 and 503. After having moved the cursor 210 to his or her desired location, the operator enters a command to generate a copy of the borderline 510 through the user interface 130. In response, the second borderline generating section 152 generates the borderline 511 by translating the borderline 510 to the location specified with the cursor 210 in accordance with the information about the first type of borderline.

Subsequently, if the operator inputs a confirmation command such as an enter command through the user interface 130, the borderline 512 is established at that location as shown in FIG. 5(c). In this manner, the borderline 512, generated by translating the borderline 510 that has been drawn by the operator, is determined.

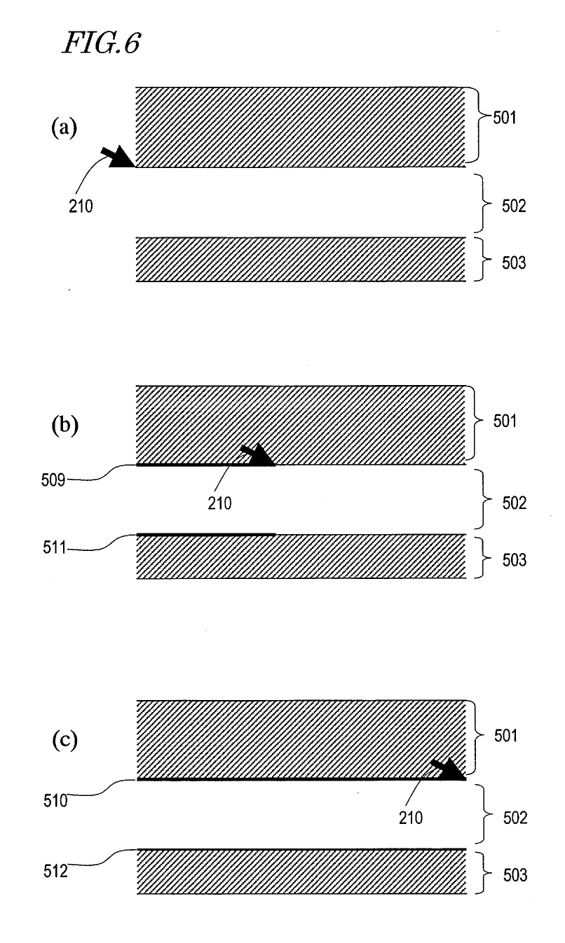

Still another procedure may also be adopted. First, as shown in FIG. 6(a), the operator puts the cursor 210 on the boundary between the tissues 501 and 502. Next, as shown in FIG. 6(b), the operator traces the boundary between the tissues 501 and 502 with the cursor 210, thereby drawing a line segment 509 indicating a first type of borderline. In the meantime, the borderline determining section 150 automatically draws a trace of points, which are located on the same acoustic line as, but at a predetermined distance from, the cursor 210, as another line segment 511. For that purpose, the first borderline generating section 153 gets the location of the cursor 210 and outputs information about that location to the borderline storage section 151. The operator inputs the interval between the locations of points to be automatically generated and the cursor 210 through the user interface 130. In the example shown in FIG. 6(b), the borderline between the tissues 502 and 503 should be generated automatically, and therefore, the interval from the boundary between the tissues 501 and 502 to the one between the tissues 502 and 503 is defined by the operator. The second borderline generating section 152 generates the line segment 511 based on the location of the cursor 210 that has been read from the borderline storage section 151 and the interval that has been entered through the user interface 130. Optionally, the interval may be determined in advance before the series of operations are started in the processing step shown FIG. 6(a).

As shown in FIG. 6(c), when the operator finishes tracing the boundary between the tissues 501 and 502 with the cursor 210, a borderline 510 is established as a first type of borderline. At the same time, a borderline 512 is automatically determined as a second type of borderline. This borderline 512 is a trace of points that are located at a predetermined distance from the location of the cursor 210 that draws the borderline 510, and therefore, has been obtained by translating the borderline 510. In this manner, a borderline 510 drawn by the operator and a borderline 512 that is a translated version of the borderline 510 are obtained.

As shown in FIG. 3, if a blood vessel is viewed on a plane that is parallel to its axis, an anterior wall and a posterior wall are seen with a vascular lumen, where blood flows, interposed, and each of the anterior and posterior walls consists of an intima, a media and an adventitia. In a target region of measurements to be done to calculate an attribute property value of the blood vessel, the boundaries between these tissues should be substantially fixed, i.e., the diameter of the vascular lumen and the thicknesses of the respective tissues should remain approximately the same, unless some disease has occurred there. That is why the boundary between the vascular flow and the intima, the boundary between the intima and media, the boundary between the media and adventitia, and the boundary between the adventitia and a surrounding tissue would be ideally roughly parallel to each other. Even if the axis of the blood vessel is not straight, the diameter of the vascular lumen and the thicknesses of the respective tissues still remain approximately the same, and therefore, the respective boundaries can be obtained by translating one of them to another.

For that reason, the second type of borderline generated by the borderline determining section 150 will agree with the actual boundary between the tissues unless the target region is adjacent to a portion with some disease. In this manner, the burden imposed on the operator by forcing him or her to trace the entire boundary and the time it will take to determine the borderlines can be both reduced. Particularly in a situation where a boundary presented on a tomographic image is not so clear, it would not cause so much trouble to the operator to specify only one point as a borderline location but it should be a lot of trouble for the operator to trace such an indefinite boundary entirely with the cursor. According to the present invention, such a burden can be lightened.

Also, although the thicknesses of the intima and media of a vascular wall and the diameter of a blood vessel could vary significantly according to the health condition of the given subject or from one person to another, the thickness of the adventitia hardly varies. It is said that at the carotid of a healthy person, the adventitia will have a thickness of approximately 0.3 mm. For that reason, unless the adventitia has any disease, the thickness of the adventitia may be defined in advance and the boundary between the adventitia and a surrounding tissue may be determined at a location that is a predetermined thickness away from the media-adventitia boundary being determined. Then, the burden imposed on the operator can be further alleviated. The adventitia preferably has a thickness of approximately 0.2 mm to approximately 0.4 mm.

The second type of borderline determined by the borderline determining section 150 does not have to be located on the boundary between the adventitia and a surrounding tissue but may also be located on any of various other boundaries. Hereinafter, an example in which the second type of borderline is determined somewhere else will be described.

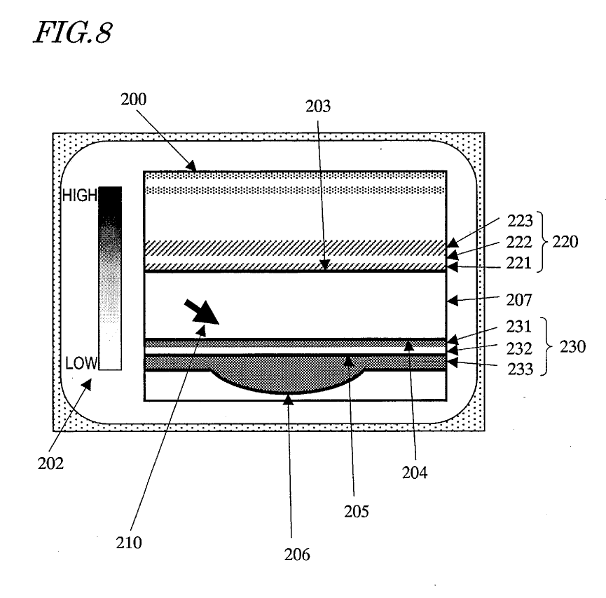

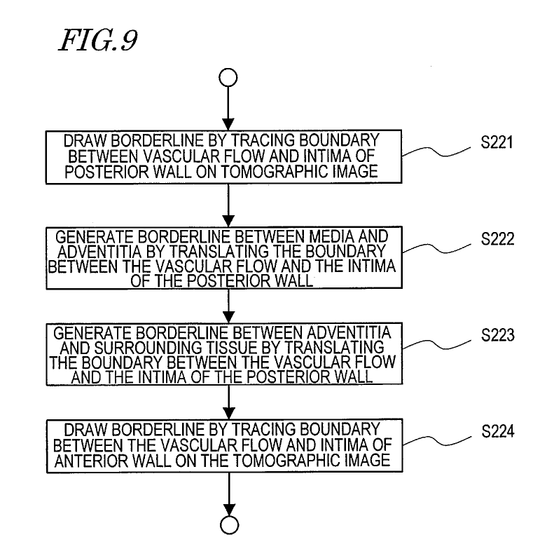

FIG. 8 illustrates a tomographic image representing a case where a boss-like disease has occurred on the outer surface of a blood vessel. FIG. 9 is a flowchart showing the procedure of determining a borderline on the tomographic image shown in FIG. 8.

First, in Step S211, by moving a cursor 210, which is displayed on the screen of the monitor 107, using the user interface 130 such as a trackball or a mouse provided for the ultrasonic diagnostic apparatus, the boundary between the vascular flow 207 and the intima 231 of the posterior wall 230 on the tomographic image 200 is traced with the cursor 210. In this manner, a borderline 204 is drawn between the vascular flow 207 and the intima 231 of the posterior wall 230 on the screen. The borderline storage section 151 stores information about the borderline 204 as a first type of borderline.

Next, in Step S212, the second borderline generating section 152 translates the borderline 204 in accordance with the information about the borderline 204 that is stored in the borderline storage section 151, thereby generating a borderline 205 on the boundary between the media 232 and adventitia 233 of the posterior wall 230 as a second type of borderline.

Subsequently, in Step S213, by moving the cursor 210 on the screen of the monitor 107 using the user interface 130, the boundary between the adventitia 233 of the posterior wall 230 and a surrounding tissue on the tomographic image 200 is traced with the cursor 210. In this manner, a borderline 206 is drawn between the adventitia 233 of the posterior wall 230 and the surrounding tissue. The borderline storage section 151 stores information about the borderline 206 as another borderline of the first type.

Finally, in Step S214, a borderline 203 between the vascular flow 207 and the intima 221 of the anterior wall 220 is drawn in the same way as in Step S213. The borderline storage section 151 stores information about the borderline 203 as still another borderline of the first type.

By following such a procedure, the burden imposed on the operator by tracing boundaries and the time it takes to determine the borderlines can be reduced by one step.

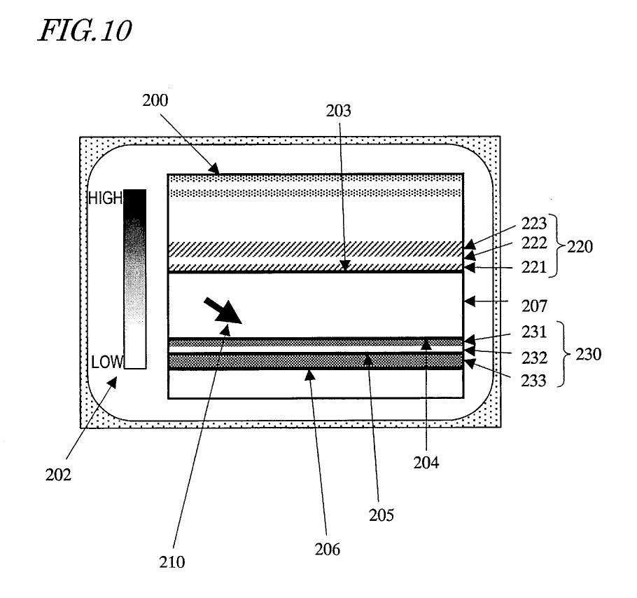

FIG. 10 illustrates a tomographic image of a healthy person's blood vessel. FIG. 9 is a flowchart showing a procedure for determining borderlines on the tomographic image shown in FIG. 10.

First, in Step S221, by moving a cursor 210, which is displayed on the screen of the monitor 107, using the user interface 130 such as a trackball or a mouse provided for the ultrasonic diagnostic apparatus, the boundary between the vascular flow 207 and the intima 231 of the posterior wall 230 on the tomographic image 200 is traced with the cursor 210. In this manner, a borderline 204 is drawn between the vascular flow 207 and the intima 231 of the posterior wall 230 on the screen. The borderline storage section 151 stores information about the borderline 204 as a first type of borderline.

Next, in Step S222, the second borderline generating section 152 translates the borderline 204 in accordance with the information about the borderline 204 that is stored in the borderline storage section 151, thereby generating a borderline 205 on the boundary between the media 232 and adventitia 233 of the posterior wall 230 as a second type of borderline.

Subsequently, in Step S223, the borderline 204 is translated again in accordance with the location information of the borderline 204 that is stored in the borderline storage section 151, thereby generating a borderline 206 on the boundary between the adventitia 233 of the posterior wall 230 and its surrounding tissue.

Thereafter, in Step S224, by moving the cursor 210 on the screen of the monitor 107 using the user interface 130, the boundary between the vascular flow 207 and the intima 221 of the anterior wall 220 on the tomographic image 200 is traced with the cursor 210. In this manner, a borderline 203 is drawn between the vascular flow 207 and the intima 221 of the anterior wall 220.

By following such a procedure, the burden imposed on the operator by tracing boundaries and the time it takes to determine the borderlines can be reduced by two steps.

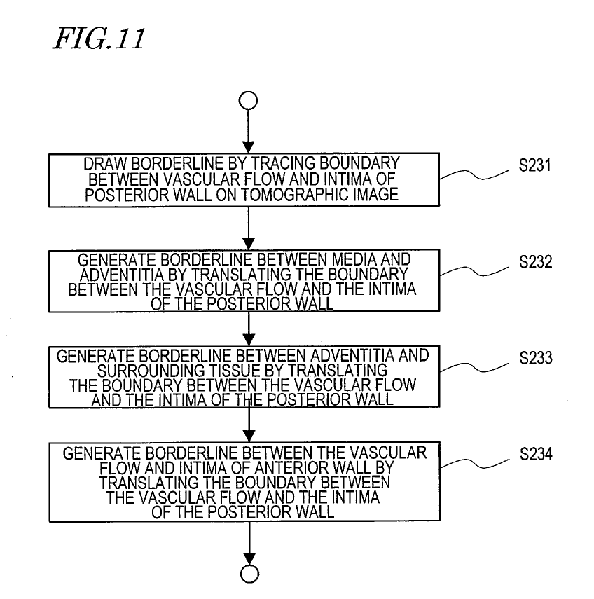

FIG. 11 is a flowchart showing another procedure for determining borderlines on the tomographic image shown in FIG. 10.

First, in Step S231, by moving a cursor 210, which is displayed on the screen of the monitor 107, using the user interface 130 such as a trackball or a mouse provided for the ultrasonic diagnostic apparatus, the boundary between the vascular flow 207 and the intima 231 of the posterior wall 230 on the tomographic image 200 is traced with the cursor 210. In this manner, a borderline 204 is drawn between the vascular flow 207 and the intima 231 of the posterior wall 230 on the screen. The borderline storage section 151 stores information about the borderline 204 as a first type of borderline.

Next, in Step S232, the second borderline generating section 152 translates the borderline 204 in accordance with the information about the borderline 204 that is stored in the borderline storage section 151, thereby generating a borderline 205 on the boundary between the media 232 and adventitia 233 of the posterior wall 230 as a second type of borderline.

Subsequently, in Step S223, the second borderline generating section 152 translates the borderline 204 again in accordance with the location information of the borderline 204 that is stored in the borderline storage section 151, thereby generating a borderline 206 on the boundary between the adventitia 233 of the posterior wall 230 and its surrounding tissue as a second type of borderline.

In the same way, in Step S234, the borderline 204 is translated once again in accordance with the location information of the borderline 204 that is stored in the borderline storage section 151, thereby generating a borderline 203 on the boundary between the vascular flow 207 and the intima 221 of the anterior wall 220 as another borderline of the second type.

By following such a procedure, the burden imposed on the operator by tracing boundaries and the time it takes to determine the borderlines can be reduced by three steps.

As described above, by translating a borderline, the burden imposed on the operator to determine at least one borderline and the time it takes to determine that borderline can be reduced.

Hereinafter, it will be described with reference to FIGS. 1(a) and 1(b) how to measure an IMT with the ultrasonic diagnostic apparatus of the present invention. First, ultrasonic waves are transmitted and received with the probe 101, thereby obtaining a series of tomographic images of a vascular wall, which are viewed on a plane parallel to its axis and which are generated consecutively at multiple points in time, and presenting them on the monitor 107 in real time. Meanwhile, the tomographic image data is stored in the memory 120. Next, in the freeze state, the data is read from the memory 120 and the best tomographic image to measure the IMT is presented on the monitor 107.

By manipulating the user interface 130, the operator moves the cursor 210 that is displayed on the screen of the monitor 107 and traces the boundary between the vascular flow and the intima of the anterior or posterior wall on the tomographic image on the monitor 107 with the cursor 210, thereby drawing a first type of borderline. The borderline storage section 151 stores information about this borderline of the first type.

Next, if the boundary between the media and adventitia is parallel to the one between the vascular flow and the intima (e.g., if there is no diseased part in the intima or in the media), then the second borderline generating section 152 translates the borderline that has been drawn on the boundary between the vascular flow and the intima, thereby generating a second type of borderline between the media and adventitia at a location corresponding to the boundary between the media and adventitia. On the other hand, if these two boundaries are not parallel to each other (e.g., if there is any diseased part in the intima), the borderline between the media and adventitia is also determined in the same way. That is to say, using the user interface 130, another borderline of the first type is drawn by moving the cursor 210 on the screen of the monitor 107 and tracing the boundary between the media and adventitia on the tomographic image with the cursor 210. Also, if the anterior and posterior walls are parallel to each other on the tomographic image, then the second borderline generating section 152 may translate the borderline that has been determined on the anterior or posterior wall, thereby generating a borderline indicating the boundary between the vascular flow and the intima of the anterior or posterior wall or between the media and adventitia thereof. Optionally, the apparatus may also be designed so as to allow the operator to determine, with the user interface 130, whether a new borderline should be generated by getting the borderline that has been drawn by the operator translated by the second borderline generating section 152 or should be generated by himself or herself using the user interface 130. The IMT can be calculated based on the interval from the borderline between the vascular flow and the intima to the one between the media and adventitia thus obtained. By adopting such a procedure, the burden and the time-consuming job imposed on the operator by tracing boundaries can be alleviated. In addition, the IMT can be calculated in a shorter time, too.

Hereinafter, it will be described how to calculate the elasticities. First, ultrasonic waves are transmitted and received with the probe 101, thereby obtaining a series of tomographic images of a vascular wall, which are viewed on a plane parallel to its axis and which are generated consecutively at multiple points in time, and presenting them on the monitor 107 in real time. Meanwhile, the tissue tracking section 105 tracks the vascular wall tissue. Using the blood pressure values provided by the blood pressure manometer 111, the elasticity calculating section 108 calculates radial elasticities and gets an image representing the distribution of the radial elasticities presented on the monitor 107. The tomographic image data and the image data representing the distribution of the radial elasticities are stored in the memories 120 and 121, respectively. The tomographic images are presented consecutively, while the image representing the distribution of the elasticities is updated once in every cardiac cycle. After having changed the modes of the ultrasonic diagnostic apparatus into the freeze state, the data is read from the memory 121 and the best image representing the distribution of the elasticities and a tomographic image synchronized with that image are read from the memory 120 and presented on the monitor 107.

By manipulating the user interface 130, the operator moves the cursor 210 that is displayed on the screen of the monitor 107 and traces the boundary between the vascular flow and the intima of the posterior wall on the tomographic image on the monitor 107 with the cursor 210, thereby drawing a first type of borderline. The borderline storage section 151 stores information about the first type of borderline thus determined.

Next, if the boundary between the adventitia of the posterior wall and its surrounding tissue is parallel to the one between the vascular flow and the intima (e.g., if there is no diseased part in the intima or in the adventitia), then the second borderline generating section 152 translates the first type of borderline that has been drawn on the boundary between the vascular flow and the intima, thereby generating a second type of borderline between the adventitia and the surrounding tissue at a location corresponding to the boundary between the adventitia and the surrounding tissue. On the other hand, if these two boundaries are not parallel to each other (e.g., if there is any diseased part in the intima or in the adventitia), the borderline between the adventitia and the surrounding tissue is also determined in the same way. That is to say, using the user interface 130, another borderline of the first type is drawn by moving the cursor 210 on the screen of the monitor 107 and tracing the boundary between the adventitia and the surrounding tissue on the tomographic image with the cursor 210.

Next, if the boundary between the vascular flow and the intima of the anterior wall is parallel to the one between the vascular flow and the intima of the posterior wall, then the second borderline generating section 152 translates the first type of borderline that has been drawn on the boundary between the vascular flow and the intima of the posterior wall, thereby generating a second type of borderline at a location corresponding to the boundary between the vascular flow and the intima of the anterior wall. On the other hand, if these two boundaries are not parallel to each other, the borderline between the vascular flow and the intima of the anterior wall is also determined in the same way. That is to say, using the user interface 130, another borderline of the first type is drawn by moving the cursor 210 on the screen of the monitor 107 and tracing the boundary between the vascular flow and the intima of the anterior wall on the tomographic image with the cursor 210.

Based on the interval from the borderline between the vascular flow and the intima of the posterior wall to the one between the adventitia and the surrounding tissue thus obtained, the initial radial thickness h0 of the overall blood vessel can be calculated. Based on the interval from the borderline between the vascular flow and the intima of the anterior wall to the one between the vascular flow and the intima of the posterior wall thus obtained, the initial radius r0 of the blood vessel can be calculated. And the circumferential elasticity can be calculated based on these values. By adopting such a procedure, the burden and the time-consuming job imposed on the operator by tracing boundaries can be alleviated. In addition, the circumferential elasticity can be calculated in a shorter time, too.

In the preferred embodiments described above, the first borderline generating section generates a line segment or a polygon as the first type of borderline by allowing the operator to draw it with the user interface 130 or specify multiple points. However, the first type of borderline may also be generated automatically. For example, a vascular flow portion and a vascular wall portion of a subject have significantly different acoustic impedances and the image signals thereof have different amplitudes. That is why the boundary between the vascular flow portion and the vascular wall portion, i.e., the boundary between the vascular flow and the intima of the vascular wall, can be detected automatically based on the amplitudes of the image signals. The first borderline generating section may detect the boundary between the vascular flow and the intima of the anterior or posterior wall based on the image signals generated by the tomographic image processing section and may determine the first type of borderline on that boundary. Also, the vascular flow portion would produce a Doppler effect in the reflected echo due to the flow of the blood. For that reason, the first borderline generating section may also detect the boundary between the vascular flow and the intima of the anterior or posterior wall by utilizing a Doppler mode and determine the first type of borderline on the boundary detected.

Meanwhile, the acoustic impedances of the intima, media and adventitia included in a vascular wall are not significantly different from each other. For that reason, it could sometimes be difficult to detect them by any of these methods. However, as described above, the boundaries between these tissues in the vascular wall are often parallel to the boundary between the vascular flow and the intima unless there is any diseased part there. In that case, if the operator can specify the boundary between the media and adventitia at an appropriate location by looking at the tomographic image, then the second borderline generating section can translate the first type of borderline, which has been automatically detected and determined, to the location specified by the operator and generate a second type of borderline there. Consequently, the burden imposed on the operator can be lightened significantly and the variations caused by the operators' decisions can be reduced greatly.

### INDUSTRIAL APPLICABILITY

The present invention is effectively applicable to an ultrasonic diagnostic apparatus that estimates the shape or attribute property value of the vascular wall of a subject.

1. An ultrasonic diagnostic apparatus for measuring a vascular wall, the apparatus comprising:
a transmitting section that drives a probe to transmit an ultrasonic wave toward a subject including a blood vessel;
a receiving section that receives an ultrasonic echo, produced by getting the ultrasonic wave reflected by the subject, at the probe to generate a received signal;
a tomographic image processing section for generating an image signal based on the received signal to present the tomographic image of the subject on a display section;
a first borderline generating section for determining a first type of borderline based on the tomographic image presented on the display section; and
a second borderline generating section for generating at least one borderline of a second type by translating the first type of borderline.

2. The ultrasonic diagnostic apparatus of claim 1, further comprising a user interface that allows an operator to specify his or her desired location on the tomographic image.

3. The ultrasonic diagnostic apparatus of claim 2, wherein the second borderline generating section generates the second type of borderline by translating the first type of borderline to the location that has been specified by the operator with the user interface in accordance with information about the first type of borderline.

4. The ultrasonic diagnostic apparatus of claim 3, wherein the first borderline generating section stores the information about the first type of borderline that has been drawn by the operator with the user interface.

5. The ultrasonic diagnostic apparatus of claim 4, wherein the first borderline generating section generates either a line segment or a polygon as the first type of borderline at the location that has been specified by the operator.

6. The ultrasonic diagnostic apparatus of claim 4, wherein the first borderline generating section stores, as information about the first type of borderline, information about a line segment that has been drawn by the operator on the tomographic image with the user interface.

7. The ultrasonic diagnostic apparatus of claim 3, wherein the first type of borderline is located on at least one boundary that is selected from a group of boundaries consisting of boundaries between the intima of the anterior and posterior walls of the blood vessel and vascular flow, boundaries between the media and adventitia of the anterior and posterior walls of the blood vessel, and boundaries between the adventitia of the anterior and posterior walls of the blood vessel and their surrounding tissue, and wherein the second type of borderline is located on at least another one of the group of boundaries.

8. The ultrasonic diagnostic apparatus of claim 7, wherein the first borderline generating section detects the boundary between the intima of the anterior or posterior wall of the blood vessel and the vascular flow based on the image signal and determines the first type of borderline on the boundary detected.

9. The ultrasonic diagnostic apparatus of claim 8, wherein the first type of borderline is located on the boundary between the intima of the anterior or posterior wall and the vascular flow and the second type of borderline is located on the boundary between the media and adventitia thereof, and
wherein the intima-media thickness of the vascular wall is calculated by reference to the first and second types of borderlines.

10. The ultrasonic diagnostic apparatus of claim 9, further comprising:
a tissue tracking section for tracking the motion of a tissue of the subject based on the received signal; and
an attribute value calculating section that receives information about the blood pressure of the subject and calculates an attribute value of the tissue based on the motion of the tissue tracked,
wherein the attribute value of at least one tissue, which is selected from the group consisting of the intima, media and adventitia of the blood vessel, is calculated with respect to the first and second types of borderlines.

11. The ultrasonic diagnostic apparatus of claim 10, wherein the first type of borderline is located on the boundary between the intima and the vascular flow and the second type of borderline is located between the adventitia and the surrounding tissue, and
wherein by calculating the radius of the blood vessel and the thickness of the vascular wall by reference to the first and second types of borderlines, a circumferential elasticity is calculated as the attribute value.

12. A method for determining the boundaries of a vascular wall using an ultrasonic diagnostic apparatus, the method comprising the step of:
generating a second type of borderline by translating a first type of borderline on a tomographic image of a subject's blood vessel that has been produced by transmitting and receiving an ultrasonic wave, the first type of borderline being determined so as to be located on at least one boundary that is selected from a group of boundaries consisting of boundaries between the intima of the anterior and posterior walls of the blood vessel and vascular flow, boundaries between the media and adventitia of the anterior and posterior walls of the blood vessel, and boundaries between the adventitia of the anterior and posterior walls of the blood vessel and their surrounding tissue, the second type of borderline being located on at least another one of the group of boundaries.

13. The method of claim 12, further comprising the step of storing, as information about the first type of borderline, information about a line segment that has been drawn on the tomographic image by the operator using a user interface,
wherein the step of generating the second type of borderline includes generating the second type of borderline based on the stored information about the first type of borderline.

14. The method of claim 12, further comprising the step of generating either a line segment or a polygon as the first type of borderline at the location that has been specified on the tomographic image of the blood vessel by the operator using the user interface.

15. The method of claim 12, further comprising the step of detecting the boundary between the intima of the anterior or posterior wall of the blood vessel and the vascular flow based on an image signal representing the tomographic image and determining the first type of borderline on the boundary detected.
